(19) United States
(12) Patent Application Publication
(10) Pub. No.: US 2011/0034896 A1
(43) Pub. Date: Feb. 10, 2011

(54) Diaper with glove portion

(76) Inventor: Yujie Bai, Arcadia, CA (US)

Correspondence Address: Raymond Sun, 12420 Woodhall Way, Tustin, CA, 92782, US

(21) Appl. No.: 12/462,817

(22) Filed: Aug. 10, 2009

(51) Int. Cl. A61F 13/84

(52) U.S. Cl. 604/385.01

(57) ABSTRACT

A diaper has an inner layer provided in an absorbent material, an outer layer corresponding in size and shape to the inner layer, and a finger receiving section provided on the rear side of the outer layer, the finger receiving section having at least one cavity that receives at least one finger of a childcare giver, with each of the at least one cavity has a sleeve-like construction and an opening. The fingers of a childcare giver's hand are inserted into the at least one cavity, then the bottom of the baby is lifted, and the diaper is slid under the bottom of the baby. The baby's bottom is then lowered onto the diaper, the fingers removed from the at least one cavity, and the diaper is then secured.

### BACKGROUND OF THE INVENTION

1. Field of the Invention

The present invention relates to diapers, and in particular, to a diaper having a feature that makes it easier to change a fresh diaper on a baby.

2. Description of the Prior Art

When a baby's diaper needs to be changed, the baby is usually laid on a changing table, pad or a bed having a flat surface. Normally, the first step of the diaper-changing process is to remove the soiled diaper and then to place the new or fresh new diaper under the baby's bottom or buttocks. To do this, the childcare giver needs to use a first hand to lift up the baby's body and the other (second) hand to place the diaper under the baby's bottom. Using conventional diapers, the childcare giver needs to hold the diaper by the fingers. This leads to the following problems:

First, the action of the second hand is limited since it is holding a diaper. Thus, the second hand cannot help the first hand during the changing process. When the baby wriggles or moves during this process, either it will be difficult to lift up and control the baby's body using the first hand alone, or it will be difficult to place the diaper to the proper position using the second hand.

Second, it is difficult to keep a soft diaper flat using only the second hand. Sometimes the diaper might be folded or wrinkled, thereby making it more difficult to properly place the diaper under the baby's bottom.

Unfortunately, these problems are continually experienced by new parents or other childcare givers. Thus, there still remains a need to provide added convenience to the diaper-changing process.

### SUMMARY OF THE DISCLOSURE

It is an object of the present invention to provide a diaper that affords added convenience to a childcare giver during the diaper-changing process.

In order to accomplish the objects of the present invention, there is provided a diaper having an inner layer provided in an absorbent material, an outer layer corresponding in size and shape to the inner layer, and a finger receiving section provided on the rear side of the outer layer, the finger receiving section having at least one cavity that receives at least one finger of a childcare giver, with each of the at least one cavity has a sleeve-like construction and an opening. The fingers of the childcare giver's hand are inserted into the at least one cavity, then the bottom of the baby is lifted, and the diaper is slid under the bottom of the baby. The baby's bottom is then lowered onto the diaper, the fingers removed from the at least one cavity, and the diaper is then secured.

### BRIEF DESCRIPTION OF THE DRAWINGS

FIG. 1 illustrates how a diaper according to the present invention can be positioned under a baby's bottom.

FIG. 2 is a perspective view of the diaper of FIG. 1.

FIG. 3 is the rear view of the diaper of FIG. 1.

FIG. 4 is a cross-sectional view taken along line 4-4 of FIG. 3.

FIG. 5 is the bottom view of the diaper of FIG. 1.

FIGS. 6-8 and 10 are rear views showing modifications that can be made to the diaper of FIG. 1.

FIG. 9 is a perspective view showing another modification that can be made to the diaper of FIG. 1.

FIG. 11 is a different perspective view of the diaper of FIG. 10.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

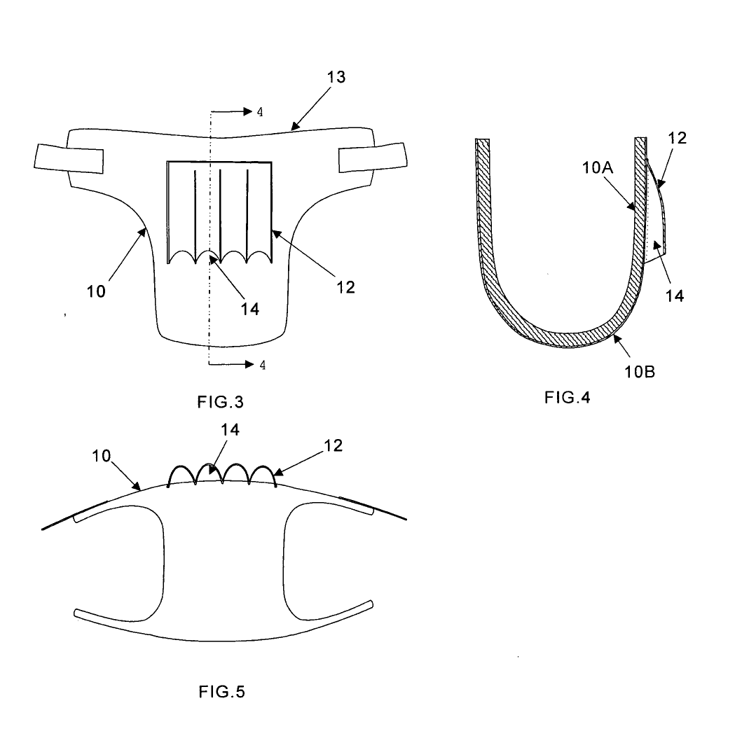

FIGS. 1-5 illustrate one embodiment of the present invention, where a disposable diaper is generally designated 10 thereof. The diaper 10 includes an inner absorbent layer 10A and an outer impervious layer 10B as best shown in FIG. 4. The inner absorbent layer 10A and the outer impervious layer 10B can be the same as for conventional diapers. A finger receiving section 12 which resembles a glove-shaped part is attached to the outer-facing surface of the outer layer 10B of the diaper 10 on the rear side of the diaper 10 (i.e., on the side of the diaper 10 that would support the baby's bottom), and is positioned near the rear upper edge 13 of the diaper 10, as best shown in FIGS. 3 and 4. The finger receiving section 12 can be attached to the outer layer 10B by stitching, hot pressing, VELCRO™ pads, adhesives or any other known connection mechanism. The finger receiving section 12 has four sleeve-like cavities 14. Each cavity 14 is configured to have a sleeve-like section with a closed upper end and opened bottom end, so as to accommodate a finger that can be inserted through the opening at the bottom end into the sleeve-like section. Thus, the purpose is allow to the diaper 10 to be worn on the four fingers of a hand, so that the hand can securely hold or carry the diaper 10 without actually using fingers to grip the diaper 10. The openings of the cavities 14 should be wide enough for the fingers to be inserted and withdrawn easily. The finger receiving section 12 can be made of the same as the material for the outer layer 10B. Such a material can have a layered construction which allows the transfer and distribution of urine to an absorbent core structure where it is locked in. For example, the diaper 10 can include an outer shell (i.e., outer layer 10B) of breathable polyethylene film or a nonwoven and film composite which prevents wetness and soil transfer. The inner layer 10A can include a first inner absorbent layer of a mixture of cellulose pulp and superabsorbent polymers for wetness, and a second layer nearest the skin of nonwoven material with a distribution layer directly beneath which transfers wetness to the first inner absorbent layer.”

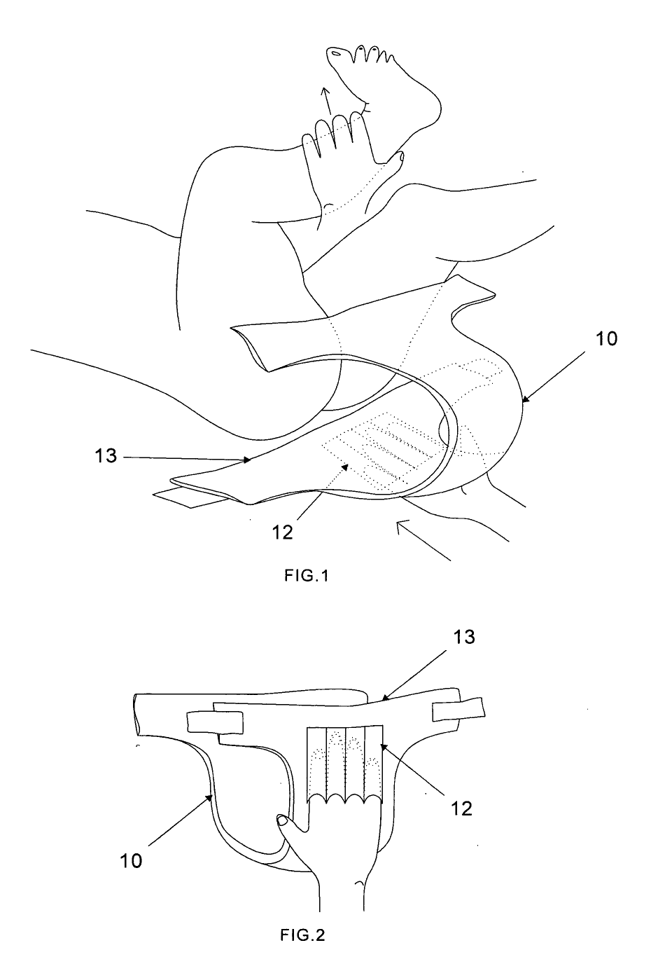

Thus, in use, the childcare giver inserts the fingers of his or her first hand into the cavities 14 of the finger receiving section 12, as shown in FIG. 2. Then the childcare giver can use the other (second) hand to lift up the baby's bottom, and the first hand wearing the diaper 10 can even be used to help lift up the baby. With the baby's bottom lifted, the fingers of the first hand can be spread out, thereby causing the diaper 10 to assume a generally flat or extended configuration, and the first hand can be slid under the baby's bottom with the diaper (see FIG. 1). At this point, the baby's bottom can be lowered on to the inner layer 10A of the rear of the diaper 10. If the baby struggles or wriggles during the positioning of the baby, the first hand can push the diaper 10 against the baby's bottom either to control the baby's wriggle or to follow the baby's movement in order to slide the diaper 10 to the desired proper position. Once the baby's bottom rests on the rear side of the diaper 10, the fingers of the first hand can be withdrawn from the cavities 14, and the diaper 10 is ready to be secured in a conventional manner.

The present invention provides an improved disposable diaper 10 that has a glove-shaped finger receiving section 12 on the back of the diaper 10 which allows the diaper 10 to be worn on a child care giver's hand as a glove to free this hand from holding the diaper 10 during the diaper-changing process. The fingers of the hand in the finger receiving section 12 can help to keep the diaper 10 extended and generally flat so that it is easier to put on the diaper for the baby, especially if baby is wriggling during the diaper-changing process. The present invention provides the following benefits:
To free the child care giver's hand from holding the diaper 10. Although it is carrying the diaper 10, this hand can still help the other hand to control the baby and to lift up the baby's body.
It helps the child care giver to better and more quickly position the diaper under the baby's bottom. When the baby wriggles, the child care giver's hand can follow the baby's wriggle to slide the diaper 10 to the proper position.
The fingers in the finger receiving section 12 can support the diaper 10 to keep it extended and generally flat so that it can be easily slid under the baby's bottom.
By essentially wearing the diaper 10 wearing on a hand, less space is required to slide the diaper 10 under the baby's bottom. It is not necessary to lift up the baby's body too high during the changing process.

The diaper 10 of the present invention can be provided in different sizes and for all people of ages. The present invention is very simple and can be implemented at low cost.

Several modifications can be made to the embodiment shown in FIGS. 1-5. For example, the finger receiving section 12 can also be used with washable diapers or other types of diapers.

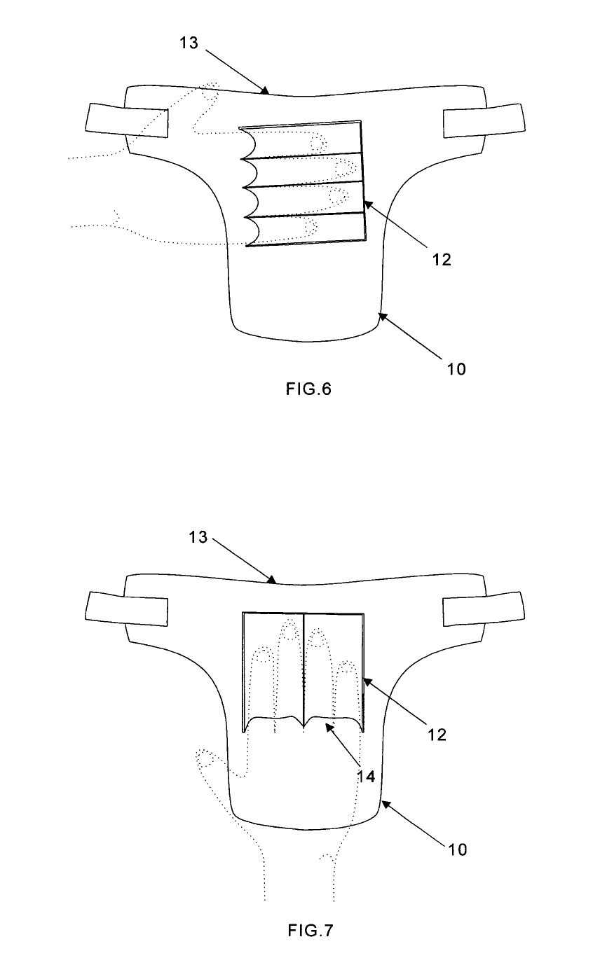

Referring to FIG. 6, the direction of the finger receiving section 12 can be rotated right or left, so that in the example shown in FIG. 6, the openings face towards the left. This modification is better suited for use with larger or heavier babies or for use with adults, who need to be cradled when lifted up. By having the openings of the finger receiving section 12 face towards one side, the caregiver can slide his or her fingers into the finger receiving section 12 from the side.

In addition, the number of cavities 14 of the finger receiving section 12 can be varied so that any number of cavities can be provided. For example, FIG. 7 shows the provision of two cavities 14, each adapted to receive two fingers. However, it is possible to provide one, three or even more than four cavities 14. In this regard, providing more than four cavities 14 allows the user to select which cavities 14 best suit his or her fingers. For example, if more than four cavities 14 are provided, a user with wider hands may prefer to use the widest cavities 14, while a user with smaller hands may prefer to use the inner cavities 14.

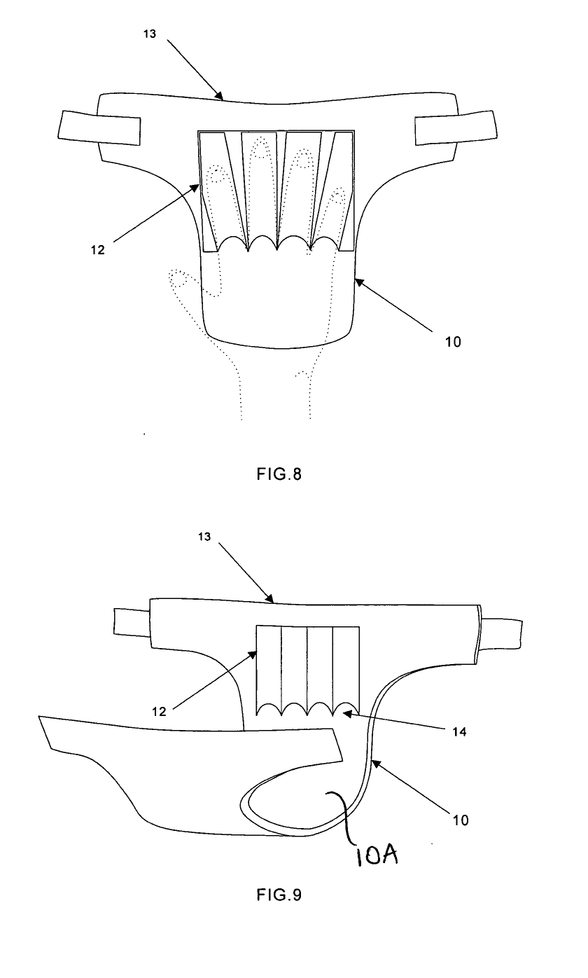

Also, as shown in FIG. 8, the cavities 14 do not need to be aligned or parallel, but can be arranged in a radial or other angled pattern. This allows the fingers to spread, thereby facilitating greater comfort and fit.

As a further alternative, the finger receiving section 12 can be provided on the inner layer 10A (see FIG. 9) by providing the section 12 in an appropriate material which matches, or is compatible, with the material of the inner layer 10A. For example, the material should be absorbent.

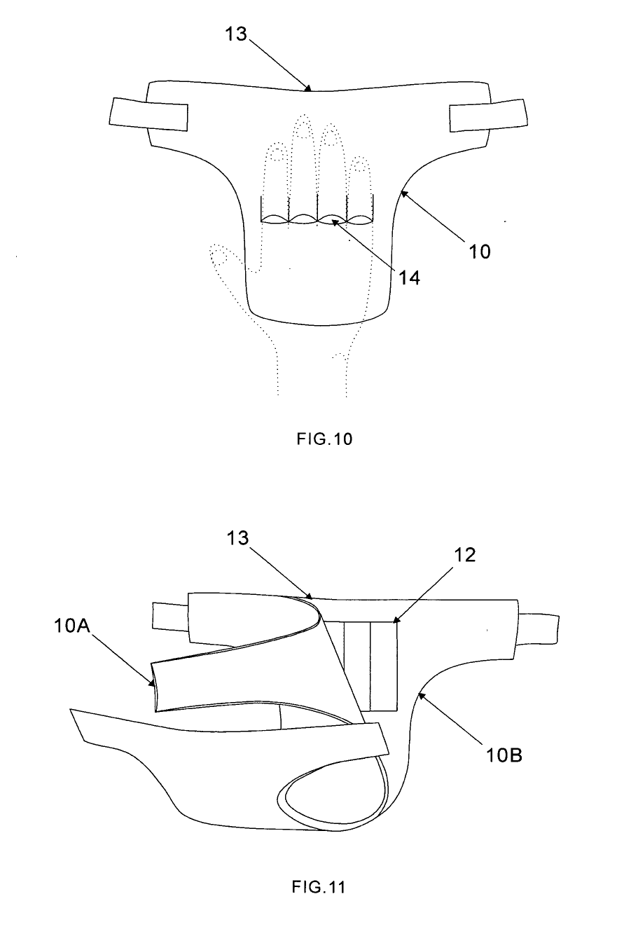

As a further example, the finger receiving section 12 can be provided on the inside of the outer layer 10B (e.g., between the layers 10A and 10B) by providing appropriate holes or openings 15 on the outer layer 10B to access the cavities 14. See FIGS. 10 and 11.

While the description above refers to particular embodiments of the present invention, it will be understood that many modifications may be made without departing from the spirit thereof. The accompanying claims are intended to cover such modifications as would fall within the true scope and spirit of the present invention.

What is claimed is:

1. A diaper, comprising:
an inner layer provided in an absorbent material;
an outer layer corresponding in size and shape to the inner layer, the outer layer having a rear side; and
a finger receiving section provided on the rear side of the outer layer, the finger receiving section having at least one cavity that receives at least one finger of a childcare giver, wherein each of the at least one cavity has a sleeve-like construction and an opening.

2. The diaper of claim 1, wherein the at least one cavity comprises a plurality of cavities, with each cavity having a sleeve-like construction and an opening that faces downwardly.

3. The diaper of claim 1, wherein the at least one cavity comprises more than four cavities.

4. The diaper of claim 1, wherein the finger receiving section is provided adjacent a top edge of the diaper.

5. The diaper of claim 1, wherein each cavity is parallel to the other cavities.

6. The diaper of claim 1, wherein each cavity is angled with respect to the other cavities.

7. The diaper of claim 1, wherein the finger receiving section is made from the same material as the outer layer.

8. The diaper of claim 1, wherein the finger receiving section is provided between the inner and the outer layers.

9. A method of changing a diaper for a baby, comprising:
removing an existing diaper from the baby;
providing a new diaper comprising:
an inner layer provided in an absorbent material;
an outer layer corresponding in size and shape to the inner layer, the outer layer having a rear side; and
a finger receiving section provided on the rear side of the outer layer, the finger receiving section having at least one cavity that receives at least one finger of a childcare giver, wherein each of the at least one cavity has a sleeve-like construction and an opening;
inserting the fingers of a childcare giver's hand into the at least one cavity;
lifting the bottom of the baby;
sliding the new diaper under the bottom of the baby;
lowering the baby's bottom onto the new diaper;
removing the fingers from the at least one cavity; and
securing the new diaper.

10. The method of claim 9, wherein further including:
spreading the fingers in the at least one cavity.
